(19) United States
(12) Patent Application Publication
(10) Pub. No.: US 2003/0012786 A1
(43) Pub. Date: Jan. 16, 2003

(54) Use of anti-TNF antibodies as drugs in treating septic disorders of anemic patients

(76) Inventors: Leah S. Teoh, Wayne, NJ (US); William T. Barchuk, Madison, NJ (US); Steven A. Fischkoff, Short Hills, NJ (US)

Correspondence Address: John D. Conway, Abbott Bioresearch Center, Inc., 100 Research Drive, Worcester, MA, 01605-4314, US

(21) Appl. No.: 10/154,025

(22) Filed: May 23, 2002

Related U.S. Application Data
(60) Provisional application No. 60/293,818, filed on May 25, 2001.

(51) Int. Cl. A61K 39/395

(52) U.S. Cl. 424/145.1

(57) ABSTRACT

The instant invention is directed to treating an anemic patient having elevated levels of IL-6 by administering a TNF antagonist. It is also directed to treating sepsis in a patient by administering a TNF antagonist.

The present invention relates to the use of TNF antagonists for treating septic disorders in anemic patients.

It is known that the term tumor necrosis factor (TNF) embraces two cytotoxic factors (TNF-α and TNF-β) which are mostly produced by activated lymphocytes and monocytes.

EP 260,610 describes, for example, anti-TNF antibodies which are said to be usable for inactivating TNF in disorders associated with an increase in TNF in the blood, such as septic shock, transpant rejection, allergies, autoimmune diseases, shock lung, coagulation disturbances or inflammatory bone disorders.

In medical textbooks, septic disorders are defined as a collective term for clinical states in which agents causing inflammation, eg. bacteria, start from a focus and reach the blood stream, which initiates a wide range of subjective and objective pathological manifestations. It is further found that the clinical picture may vary widely depending on the type of causative agent, the responsivity of the body, the primary focus and the varying involvement of organs.

A number of cytokines have been suggested to be involved in the complex pathophysiological process of sepsis. TNF in particular is, on the basis of data from animal experiments (Beutler et al,. Science 229 (1985) 869-871), ascribed an important role in septic shock.

This has led to clinical studies being carried out on the treatment of sepsis patients with anti-TNF antibodies.

In a published multicenter phase II study on the treatment of severe septicemia with a murine monoclonal anti-TNF antibody it was found that the overall population (80 patients) did not benefit in terms of survival rate from the treatment with the antibody. Only the patients with elevated circulating TNF concentrations appeared to benefit in terms of the probability of survival from high-dose anti-TNF antibody administration (C. J. Fisher et al., Critical Care Medicine, Vol. 21, No. 3, pages 318-327). There is also a reference in this study to a correlation between the plasma levels of TNF and IL-6.

The part played by the cytokine interleukin-6 (IL-6) in sepsis is unclear and contradictory. Elevated levels of IL-6 have been found in the serum of sepsis patients (Hack et al., Blood 74, No. 9, (1989) 1704-1710).

Waage describes a correlation between the concentrations of the cytokines IL-6 and IL-8 with the severity of the shock, although they had no effect, either alone or in combination with TNF, in terms of mortality, on the development of a shock syndrome (Waage in “Tumor Necrosis Factors”, ed. B. Beutler, Raven Press, New York, 1992, pages 275-283).

Some scientists have ascribed a beneficial role to IL-6 in septic shock because IL-6 inhibits, in the form of negative feedback control, the LPS-induced TNF production (Libert et al. in “Tumor Necrosis Factor Molecular and Cellular Biology and Clinical Relevance”, ed. W. Fiers, Karger, Basle, 1993, pages 126-131).

WO 95/00291 discloses TNF antagonists as medicines for treating sepsis in patients in whom the serum levels of interleukin-6 are 500 pg/ml or more.

WO 99/21582 discloses TNF antagonists as medicines for treating sepsis in patients in whom the serum levels of interleukin-6 are increasing at the time of treatment.

It has now been found that a certain sub-set of patients having elevated IL-6 levels and anemia respond advantageously to therapy. The treatment of septicemia with TNF antagonists is particularly successful according to this invention, for example measured by a significant reduction in mortality, when the septicemic patients who are treated are anemic and have II-6 levels of about 1000 pg/ml or more at the start of treatment.

The serum concentrations of IL-6 can be determined by conventional detection methods such as RIA or ELISA. An example of a very suitable detection system is the IL-6 EASIA supplied by Medgenix. The concentration of IL-6 can also be determined in an activity assay in which, for example, C-reactive protein is assayed.

Suitable TNF antagonists are anti-TNF antibodies, TNF receptors or soluble fragments thereof, TNF-binding proteins or those TNF derivatives which still possess TNF receptor binding but no longer have any TNF activity. TNF antagonists of these types have the characteristic that they trap TNF which has already been produced and do not allow it to reach the TNA receptor or that they compete with the TNF for the receptor.

However, TNF antagonists which prevent the formation or release of TNF are also suitable for the use according to the invention. Substances of this type inhibit, for example, TNF gene expression or the release of TNF from precursor forms. Examples of suitable TNF antagonists are inhibitors of TNF convertase.

TNF-antagonistic activities have been described, for example, for xanthine derivatives, glucocorticoids, prostaglandin E 2, thalidomide, interleukin-4, interleukin-10, granulocyte stimulating factor (G-CSF), cyclosporin and α-antitrypsin. Thus compounds of these types are also suitable as TNF antagonists.

The TNF antagonists suitable for the use according to the invention are described, for example, by Mariott et al., DDT, Vol. 2, Nol &, July 18997 and in the literature cited therein.

Anti-TNF antibodies and fragments thereof are particularly preferrred for the use according to the invention.

The anti-TNF antibodies suitable for the use according to the invention are know (EP 260,610, EP 351,789, EP 218,868). It is possible to use both polyclonal and monoclonal antibodies. Also suitable in addition are TNF-binding antibody fragments such as Fab or F(ab′)₂fragments or single-chain Fv fragments.

Humanized or human anti-TNF antibodies or their TNF-binding fragments are also very suitable because these molecules ought not to cause any anti-mouse antigenicity in human patients.

It is also possible to use mixtures of various anti-TNF antibodies or of anti-TNF antibodies and TNF receptor fragments as active ingredients.

The present invention also includes pharmaceutical compositions that contain nontoxic, inert pharmaceutically suitable carriers and the anti-TNF antibodies, and process for producing these compositions.

The anti-TNF antibodies are formulated in a way customary for biotechnologically produced active ingredients, as a rule as a liquid formulation of lyophilisate. The pharmaceutical compositions mentioned above are produced in a conventional way by methods know to one of ordinary skill in the are, for example, by mixing the active ingredient(s) with the carrier(s).

In general it has proven advantageous to administer the active ingredient suitable for the use of the present invention in total amounts of about 0.1 to about 100, preferable 0.1 to 10, mg/kg of body weight every 24 hours, where appropriate in the form of several individual doses or as continuous infusion and, where appropriate, over a treatment period of several days to achieve the desired results. Administration can take place as brief intravenous infusions of the single doses or as continuous long-term infusion of the daily dose over 24 hours. A single dose preferably contains the active ingredient(s) in amounts of about 0.1 to about 10 mg/kg of body weight. However, it may be necessary to deviate from the stated dosages, specifically depending on the age and size of the patient to be treated and on the nature and severity of the fundamental disorder affecting the patient, the type of composition and of administration of the drug, and the period or interval over which administration takes place.

The invention is illustrated further in the following Example.

### EXAMPLE

Treatment of septicemic patients with a murine anti-TNF antibody fragment (F(ab′)₂), called Mab 195F (INN: Afelimomab)

A total of 2634 patients with severe sepsis were treated in a multicenter clinical study with anti-TNF antibody fragment (afelimomab) or with placebo. The patients were assigned to either the group receiving afelimomab or placebo by random. The therapy was given in addition to the standard therapy for septicemic patients and consisted of the administration as a brief infusion of 1 mg/kg of afelimomab or placebo every eight hours for three days, a total of nine treatments.

Of these patients, 998 had a serum level of IL-6 of about 1000 pg/ml or above at the start of treatment.

A decrease in mortality was obtained in the group of patients that had serum levels of IL-6 of about 1000 pg/ml and above and also had a low value for hemoglobin, hematocrit or red blood cell count. More specifically, in the group of patients who had a hemoglobin value less than or equal to 11 g/dl, the administration of afelimomab was significantly effective in reducing the level of mortality. Likewise, in the group of patients having a hematocrit value of less that 35.5%, the administration of afelimomab was significantly effective in reducing the level of mortality. In patients having a red blood cell count of less than 3.5×10⁹/l, the administration of afelimomab was significantly effective in reducing the level of mortality. The results are set forth below in Table I.

| Effect | Odds Ratio | P-value |
|---|---|---|
| Hemoglobin ≦ 11 g/dl | 1.985 | 0.0002 |
| Hematocrit < 35.5% | 1.676 | 0.0012 |
| Red Blood Cell Count < | 2.153 | 0.0001 |
| 3.5 × 10⁹/l | | |

In general, the low levels found for hemoglobin, hematocrit and red blood cell count are all indicative of anemia in a patient.

The result of this clinical study demonstrates that the treatment of severe sepsis with anti-TNF antibodies is particularly successful when the sepsis patients who are treated are anemic.

1. A method of treating anemic patients having elevated serum levels of interleukin-6 which comprises administering a therapeutically effective amount of a TNF antagonist to said patient.

2. The method of claim 1 wherein the wherein the serum level of interleukin-6 is above about 1000 pg/ml.

3. The method of claim 1 wherein the TNF antagonist is a monoclonal anti-TNF antibody.

4. The method of claim 3 wherein the monoclonal anti-TNF antibody is a F(ab′)₂ fragment.

5. The method of claim 4 wherein the F(ab′)₂ fragment is afelimomab.

6. The method of claim 1 wherein the patient has a hemoglobin level less than or equal to 11 g/dl.

7. The method of claim 1 wherein the patient has a hematocrit level below 35.5%.

8. The method of claim 1 wherein the patient has a red blood cell count below 3.5×10⁹/l.

9. A method of treating sepsis in an anemic patient which comprises administering a therapeutically effective amount of a TNF antagonist to said patient.

10. The method of claim 6 wherein the wherein the serum level of interleukin-6 is above about 1000 pg/ml.

11. The method of claim 6 wherein the TNF antagonist is a monoclonal anti-TNF antibody.

12. The method of claim 8 wherein the monoclonal anti-TNF antibody is a F(ab′)₂ fragment.

13. The method of claim 9 wherein the F(ab′)₂ fragment is afelimomab.

14. The method of claim 1 wherein the patient has a hemoglobin level less than or equal to 11 g/dl.

15. The method of claim 1 wherein the patient has a hematocrit level below 35.5%.

16. The method of claim 1 wherein the patient has a red blood cell count below 3.5×10⁹/l.
